(19) United States
(12) Patent Application Publication
(10) Pub. No.: US 2008/0097374 A1
(43) Pub. Date: Apr. 24, 2008

(54) Inflatable shaped balloons

(76) Inventors: Joseph E. Korleski, Newark, DE (US); Jeffrey Towler, Wilmington, DE (US)

Correspondence Address: Bridget Sciamanna;W. L. Gore & Associates, Inc., 551 Paper Mill Road, P.O. Box 9206, Newark, DE, 19714-9206, US

(21) Appl. No.: 11/500,837

(22) Filed: Aug. 7, 2006

(51) Int. Cl. A61M 29/02; A61M 25/10

(52) U.S. Cl. 604/500; 604/103.1

(57) ABSTRACT

An inflatable device with at least two helically wrapped passes forming a tube and an inflation means connected to said tube is provided. The tube is configured and joined in at least one area forming a low profile continuous inflatable device with multiple cross-sectional segmented areas.

### BACKGROUND OF THE INVENTION

The present invention relates to inflatable balloons and more particularly to inflatable shaped balloon catheters for medical procedures.

Balloon catheters are well known in the art. Such catheters are employed in a variety of medical procedures, including dilation of narrowed blood vessels, placement of stents and other implants, temporary occlusion of blood vessels, vertebral compaction, vertebrae compression and compaction, vascular uses, and other medical procedures.

In a typical application, the balloon is advanced to the desired location in the vascular system. The balloon is then pressure-expanded in accordance with a medical procedure. Thereafter, the pressure is removed from the balloon, allowing the balloon to contract and permitting removal of the catheter. It is to be appreciated that the balloon must be formed of a material which is readily pressure-expanded, yet will also readily contract upon removal of the inflation pressure.

Procedures such as these are generally considered minimally-invasive, and are often performed in a manner which minimizes disruption to the patient's body. As a result, catheters are often inserted from a location remote from the region to be treated. For example, during angioplasty procedures involving coronary vessels, the balloon catheter is typically inserted into the femoral artery in the groin region of the patient, and then advanced through such vessel into the coronary region of the patient. These catheters typically include some type of radiopaque marker to allow the physician performing the procedure to monitor the progress of the catheter through the body. However, because the balloon portion of the catheter is not visible to the physician, the balloon may be over inflated without the physician's awareness. This is particularly concerning when large diameter balloons are employed in medical procedures because the maximum stress of the inflated balloon material can more easily be exceeded causing the balloon to rupture or burst.

There are two main forms of balloon catheter devices, compliant and non-compliant. Angioplasty catheters employ a balloon made of relatively strong but generally inelastic material (e.g., polyester) folded into a compact, small diameter cross section. These relatively stiff catheters are used to compact hard deposits in vessels. Due to the need for strength and stiffness, these devices are rated to employ high inflation pressures, usually up to about 8 to 12 atmospheres depending on rated diameter. They tend to be self-limiting as to diameter in that they will normally distend up to the rated diameter and not distend appreciably beyond this diameter until rupture due to over-pressurization. While the inelastic material of the balloon is generally effective in compacting deposits, it tends to collapse unevenly upon deflation, leaving a flattened, folded balloon substantially larger in cross section than the balloon was prior to inflation. Because of the tendency of these types of balloons to assume a flattened cross section upon deflation, the deflated maximum width tends to approximate a dimension corresponding to one-half of the rated diameter multiplied by pi. This enlarged, folded balloon may be difficult to remove, especially from small vessels. Further, because these balloons are made from inelastic materials, their time to complete deflation is inherently slower than elastic balloons.

By contrast, embolectomy catheters employ a soft, very elastic material (e.g., natural rubber latex) as the balloon. These catheters are employed to remove soft deposits, such as thrombus, where a soft and tacky material such as latex provides an effective extraction means. Latex and other highly elastic materials generally will expand continuously upon increased internal pressure until the material bursts. As a result, these catheters are generally rated by volume (e.g., 0.3 cc) in order to properly distend to a desired size. Although relatively weak, these catheters do have the advantage that they tend to readily return to their initial size and dimensions following inflation and subsequent deflation. The weak nature of the elastomer material used in these types of balloon catheters has restricted their use to small diameter balloon applications; typically less than 4 to 5 mm diameter. The stress generated in the inflatable balloon material is defined as hoop stress and is a function of the product of the inflation pressure and the inner diameter of the inflated balloon, divided by the wall thickness of the inflated balloon. Accordingly, the hoop stress increases linearly with increasing balloon diameter. Therefore, there have been efforts to reinforce embolectomy elastic balloon catheters.

Some catheter balloons constructed of both elastomeric and non-elastomeric materials have been described previously. U.S. Pat. No. 4,706,670 describes a balloon dilatation catheter constructed of a shaft made of an elastomeric tube and reinforced with longitudinally inelastic filaments. This device incorporates a movable portion of the shaft to enable the offset of the reduction in length of the balloon portion as the balloon is inflated. One improved balloon is disclosed in U.S. Pat. No. 4,706,670 teaching reinforcing filaments in a balloon portion. U.S. Pat. No. 5,647,848 teaches a structure formed of helically extending fibers, including bundles of continuous monofilaments, aramide, polyethylene, steel, polyester, glass, carbon, and ceramics. The fibers are positioned in an elastomer such that the fibers lie at an angle which is less than a neutral angle of 54.73 degrees relative to the axis of the balloon when the balloon is unpressurized. With the utilization of rigid fibers, the balloon will be non-compliant in its fully inflated state. The difference in rigidity, although desirable with respect to independent movement of the components of the balloon, can introduce unwanted torsional moments into the elastomeric balloon depending upon the construction of the balloon and fibers.

Some medical procedures require the use of a relatively large diameter balloon (size), such as valvuloplasty, aortic stent graft deployment, pediatric coarctation, sizing balloon for ASD/PFO (atrial septal defect/patent foramen ovale), endocardial procedures, stent deployment and vertebrae compression and compaction, including kyphoplasty and would greatly benefit from a balloon with a small uninflated diameter that would return to that initial size and dimensions following inflation and subsequent deflation. The means for reinforcing the elastic balloon catheters to date have not addressed both the low profile and high burst pressure requirements for large diameter balloon applications. Accordingly, there is a need in the art for a large diameter balloon that can maintain a shape profile upon inflation and that can withstand high inflation pressure.

### SUMMARY OF THE INVENTION

The present invention provides a shaped balloon suitable for use with a catheter in a variety of surgical procedures. The shaped balloons of the present invention can be made to reach large outer diameters and sustain high inflation pressures while maintaining their shape. These balloons exhibit retraction approximating their pre-inflation shape upon deflation. In addition, the shaped balloons of the present invention can be made to provide perfusion flow through the center or open region of the inflated balloon. Furthermore, the shaped balloon of the present invention can be attached to a catheter for the purpose of delivering a device or treatment element such as a radioactive element through the center portion of the inflated balloon; alternatively, two balloons can be used to center a device or treatment element in the center of a vessel, tube or orifice.

The shaped balloon of the present invention comprises a plurality of wrapped composite film layers formed into a hollow body that is configured into a shaped balloon and fixed in that shape. Means are provided for fixing the shape including heat setting and over-wrapping the configured shaped balloon with an outer configuration layer of material. In a preferred embodiment, the composite film comprises a high strength porous reinforcing polymer and a continuous phase of polymer coated onto at least one side of the porous reinforcing polymer. In a more preferred embodiment, the porous reinforcing polymer is expanded polytetrafluoroethylene (ePTFE) membrane. The ePTFE membrane is coated with an elastomer, wherein the elastomer is imbibed into and fills the pores of the ePTFE membrane.

### DESCRIPTION OF THE DRAWINGS

FIG. 1 shows a schematic distal end view representation of an inflated folded shaped balloon with an open region.

FIG. 2 shows a schematic distal end view representation of an inflated folded shaped balloon with no open region.

FIG. 3 shows a cross-section view of a composite film with two polymer coating layers on the porous reinforcing polymer.

FIG. 4 shows a cross-section view of a composite film with one polymer coating layer on the porous reinforcing polymer.

FIG. 5 shows a cross-section view of a composite film with two polymer coating layers and polymer imbibed throughout the porous reinforcing polymer.

FIG. 6 shows a schematic representation of the composite film helically wrapped around a core wire.

FIG. 7 shows a schematic representation of a second wrap layer forming the non-distensible regions.

FIG. 8 shows a schematic representation of many non-distensible regions along the length of a balloon.

FIG. 9 shows a schematic representation of many non-distensible regions along the length of an inflated balloon.

FIG. 10 shows a schematic representation of an inflated folded shaped balloon with an outer configuration layer.

FIG. 11 shows a schematic representation of an inflated folded shaped balloon attached to a catheter and deployed in a vessel.

FIG. 12 shows a schematic representation of an inflated folded shaped balloon attached to a catheter and deployed in a vessel and perfusive flow.

FIG. 13 shows a schematic representation of two inflated folded shaped balloons attached to a catheter and deployed in a vessel with a treatment element secured between the two balloons.

FIG. 14 shows a schematic representation of a spirally shaped balloon with an outer configuration layer.

FIG. 15 shows a schematic representation of a spirally shaped balloon with an open region.

FIG. 16 shows a hollow center multi-chambered balloon.

FIG. 17 shows a hollow center coiled balloon.

FIG. 18 shows a coiled balloon.

FIG. 19 shows a spiral shaped balloon with treatment device

### DETAILED DESCRIPTION OF THE INVENTION

The present invention provides a shaped balloon catheter for use in a variety of surgical procedures. The shaped balloon is able to obtain large outer diameters while sustaining high inflation pressures. The balloon may be attached to a catheter and is comprised of at least two passes of a balloon material.

The balloon material of the present invention is a composite film comprising a porous reinforcing layer and a continuous polymer layer. The porous reinforcing polymer layer is preferably a thin, strong porous membrane that can be made in sheet form. The porous reinforcing polymer can be selected from a group of polymers including, but not limited to, olefin, PEEK, polyamide, polyurethane, polyester, polyethylene, and polytetrafluoroethylene. The porous reinforcing polymer is preferably expanded polytetrafluoroethylene (ePTFE) made in accordance with the teachings of U.S. Pat. No. 5,476,589 or U.S. patent application Ser. No. 11/334,243 to Bacino.

In one preferred embodiment, the ePTFE membrane is anisotropic such that it is highly oriented in the one direction. An ePTFE membrane with a matrix tensile value in one direction of greater than 690 megapascals is preferred, and greater than 960 megapascals is even more preferred, and greater than 1,200 megapascals is most preferred. The exceptionally high matrix tensile value of ePTFE membrane allows the composite material to withstand very high hoop stress in the inflated balloon configuration. In addition, the high matrix tensile value of the ePTFE membrane makes it possible for very thin layers to be used which reduces the deflated balloon profile. A small profile is necessary for the balloon to be able to be positioned in small arteries or veins or orifices. In order for balloons to be positioned in some areas of the body, the balloon catheter must be able to move through a small bend radius, and a thinner walled tube is typically much more supple and capable of bending in this manner without creasing or causing damage to the wall of the vessel.

In another preferred embodiment, the ePTFE membrane is relatively mechanically homogeneous. The mechanically balanced ePTFE membrane can increase the maximum hoop stress that the composite film made therefrom can withstand.

The continuous polymer layer of the present invention is coated onto at least one side of the porous reinforcing polymer. The continuous polymer layer is preferably an elastomer, such as, but not limited to, aromatic and aliphatic polyurethanes including copolymers, styrene block copolymers, silicones, preferably thermoplastic silicones, fluoro-silicones, fluoroelastomers, THV and latex. In one embodiment of the present invention, the continuous polymer layer is coated onto only one side of the porous reinforcing polymer. The continuous polymer layer is coated onto both sides of the porous reinforcing polymer. In a preferred embodiment, the continuous polymer layer is imbibed into the porous reinforcing polymer and the imbibed polymer fills the pores of the porous reinforcing polymer.

The continuous polymer layer can be applied to the porous reinforcing polymer through any number of conventional methods including, but not limited to, lamination, transfer roll coating, wire-wound bar coating, reverse roll coating, and solution coating or solution imbibing. In a preferred embodiment, the continuous polymer layer is solution imbibed into the porous reinforcing polymer. In this embodiment, the continuous polymer layer is dissolved in a suitable solvent and coated onto and throughout the porous reinforcing polymer using a wire-wound rod process. The coated porous reinforcing polymer is then passed through a solvent oven and the solvent is removed leaving a continuous polymer layer coated onto and throughout the porous reinforcing polymer. In some cases, such as when silicone is used as the continuous polymer layer, the coated porous reinforcing polymer may not require the removal of solvent. In another embodiment, the continuous polymer layer is coated onto at least one side of the porous reinforcing polymer and maintained in a “green” state where it can be subsequently cured. For example, an ultraviolet light (UV) curable urethane may be used as the continuous polymer layer and coated onto the porous reinforcing polymer. The composite film comprising the porous reinforcing polymer and the UV curable urethane continuous polymer layer can then be wrapped to form at least one layer of the balloon and subsequently exposed to UV light and cured.

An individual pass is comprised of one or more layers of balloon material which are laid at a similar angle in relation to the longitudinal axis of the balloon. A layer is considered to be one thickness of balloon material which may be wrapped, folded, laid or weaved over, around, beside or under another thickness. A longitudinal pass comprises a distinctive layer or series of layers of material which are wound to form a region or area distinct from surrounding or adjoining parts. For instance a pass may comprise multiple layers of balloon material wrapped at a 90 degree angle relative to the longitudinal axis. This exemplary pass may then be flanked by layers of balloon material wrapped at dissimilar angles in relation to the longitudinal axis, thus defining the boundary of the pass.

A pass of balloon material may be oriented helically, radially or longitudinally. By layers of balloon material it is meant to include pieces, threads, layers, filaments, membranes, or sheets of suitable balloon material. In helically oriented layers, the material is oriented so to form a balanced force angle in relation to each other upon inflation. The layers may further be wound upon themselves in subsequent passes.

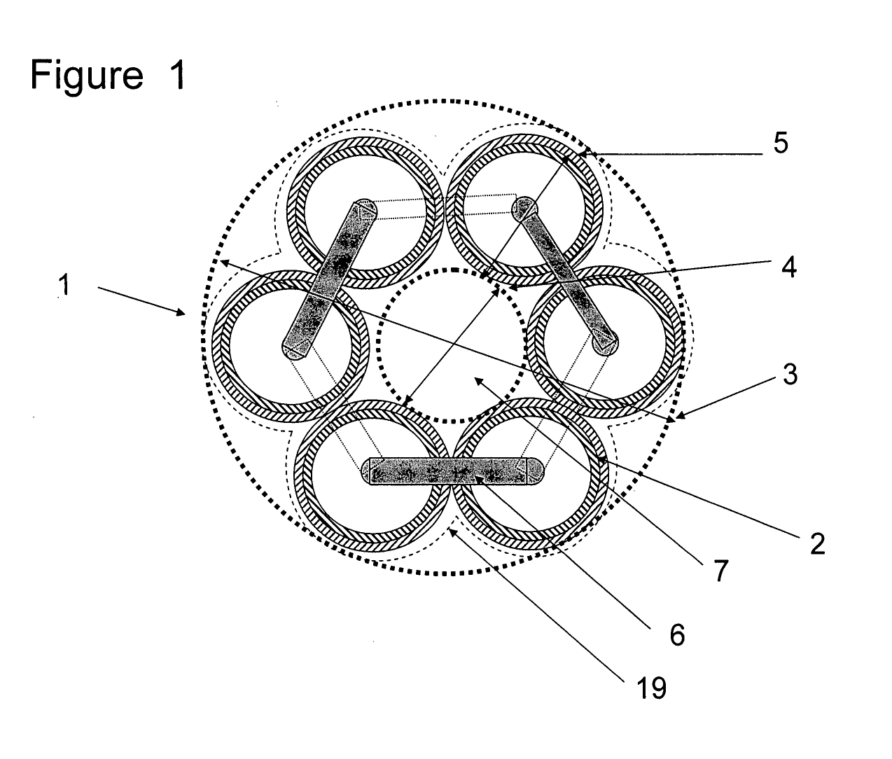

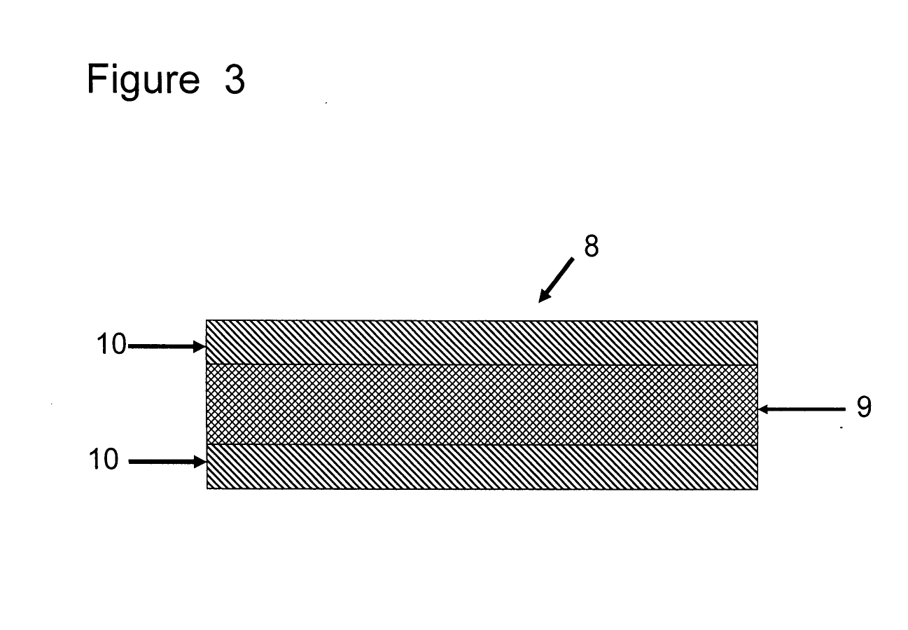

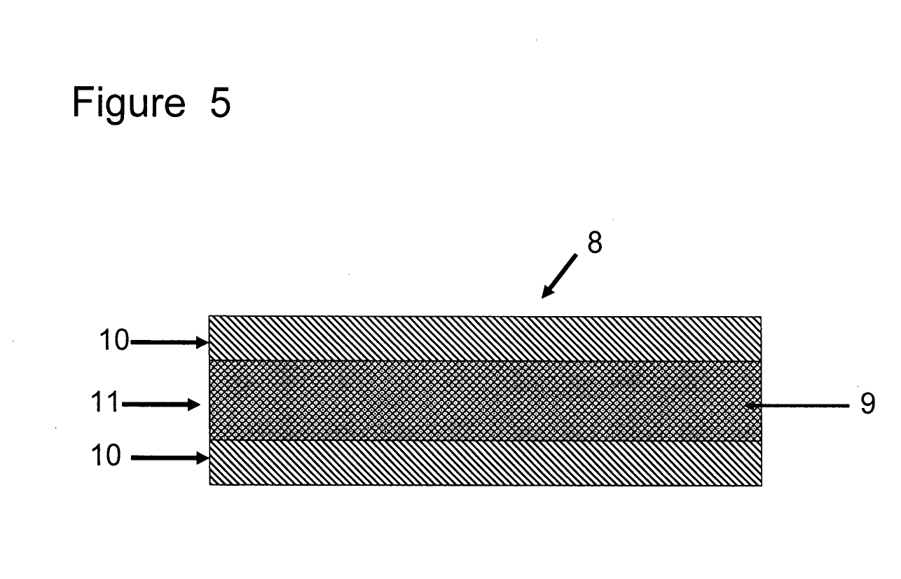

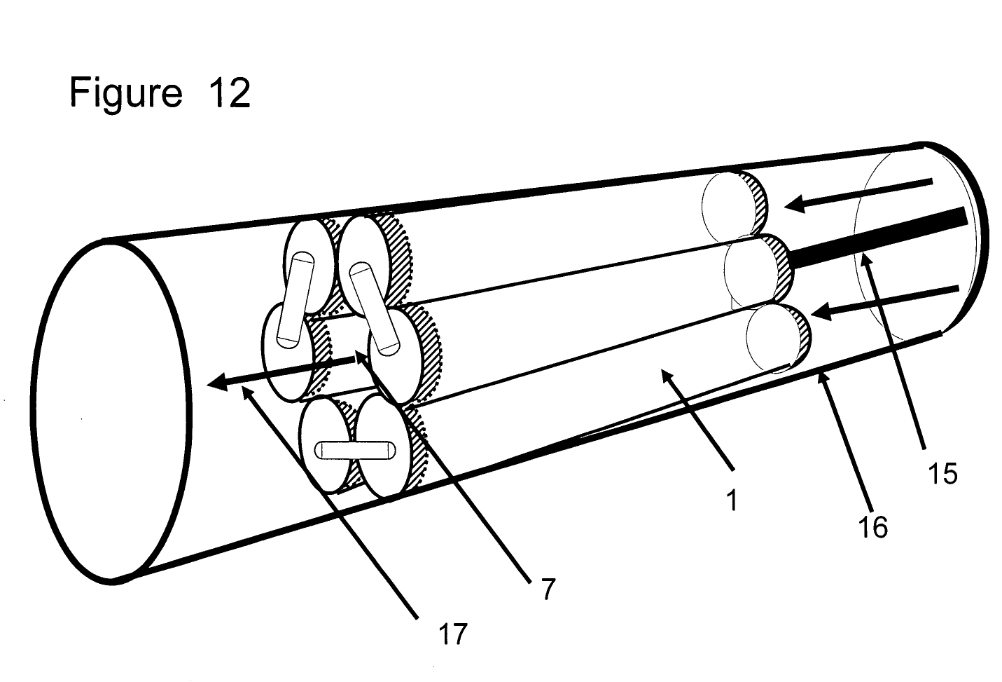

The inflated shaped balloon 1, as depicted in FIG. 1, can be made to have a much larger outer diameter 3 than what would normally be achievable with an angioplasty type balloon configured in a traditional tube. The inflated shaped balloon 1 withstands high inflation pressure at larger outer diameter 3, because it is comprised of a number of smaller balloon segments. By “shaped balloon” it is meant that a single tubular balloon is configured through folding, twisting, wrapping, winding or other mechanical manipulation of the tubular balloon into a desired configuration, and then restrained into said desired configuration so that upon inflation the single tubular balloon inflates into the desired configuration or shape. In a wrapped balloon, at least one of the wrapped balloon material passes 2 is in contact with the outer configuration layer 19. Inflatable devices such as inflatable shaped balloons comprising a non-configured tube with at least two helically wrapped layers may be fixed into an inflatable form which is substantially shorter than the non-configured tube. By “substantially shorter” it is meant that the extended non-configured tube is at least 20% longer than the fixed inflatable form, and in many embodiments, greater than twice the non-configured tube length. The inflatable form is an inflatable shaped balloon with an outer diameter 3 as depicted in FIGS. 1, 2, and 14-18 and is defined by the length of a line running through the center of the balloon and spanning the distance between the outermost walls of the inflated shaped balloon. The inner diameter 4 of the inflated shaped balloon 1 is shown as smaller than the outer diameter 3 of the inflated shaped balloon 1. The inflated balloon segment diameter 5 is smaller than the outer diameter 3 of the inflated shaped balloon 1 and is measured from the outer wall of the inflated shaped balloon 1 to the inner diameter 4 as shown. Multiple non-distensible regions 6 may be present. An open region 7 is also achievable which allows passage through the balloon. Not all shaped balloons will comprise a hollow center forming the inner diameter 4. An outer configuration layer 19 is used to hold the balloon in a desired shape. In addition, the inflated shaped balloon 1 can withstand high inflation pressures and still achieve a large outer diameter 3 because the balloon is made out of a composite film 8 as shown in FIGS. 3 to 5 that incorporates a porous reinforcing polymer 9 and a continuous polymer layer 10 on at least one side of the porous reinforcing polymer 9. As shown in FIG. 5, the composite film may further comprise an imbibed polymer 11 which infiltrates the pores or spaces of the porous reinforcing layer. In addition, perfusive flow can be achieved through the open region 7 of the shaped balloon 1 as depicted in FIG. 12, or in between the spaces of the segments. The preferred porous reinforcing polymer of the present invention is an ePTFE membrane. A very thin, strong, and anisotropic membrane is desirable to enable the balloon to achieve large diameters and sustain high inflation pressures. The ePTFE membrane provides the reinforcement necessary to withstand the high hoop stress created when large diameter balloons are inflated to high pressure. An anisotropic membrane is one which is highly oriented to provide strength in one direction.

The shaped balloon of the present invention comprises a plurality of wrapped balloon passes 2. The passes are made up of one or more layers of film or membrane wrapped at similar angles. The layers may be built up upon each other to a desired thickness. The wrap layers are comprised of film or membrane. The composite film 8 of the present invention may comprise a porous reinforcing polymer 9 and a continuous polymer layer 10 as depicted in FIGS. 3 to 5. The porous reinforcing polymer layer 9 is preferably a thin, strong, porous membrane that can be made in sheet form. The porous reinforcing polymer 9 can be selected from a group of polymers including but not limited to: olefin, PEEK, polyamide, polyurethane, polyester, polyethylene, and polytetrafluoroethylene. In one preferred embodiment, the porous reinforcing polymer 9 is expanded polytetrafluoroethylene (ePTFE) made in accordance with the teachings of U.S. Pat. No. 5,476,589 to Bacino incorporated herein by reference with the ePTFE. It is unusually strong, is unusually thin, has unusually small pore sizes, but a very high air flow-through. It has a pore size between 0.05 and 0.4 micrometers; a bubble point between 10 and 60 psi; a pore size distribution value between 1.05 and 1.20; a ball burst strength between 0.9 and 17 pounds/force; an air flow of between 20 Frazier and 10 Gurley seconds; a thickness between 1.0-25.4 micrometers; and a fiber diameter ranging between 5 and 200 nm.

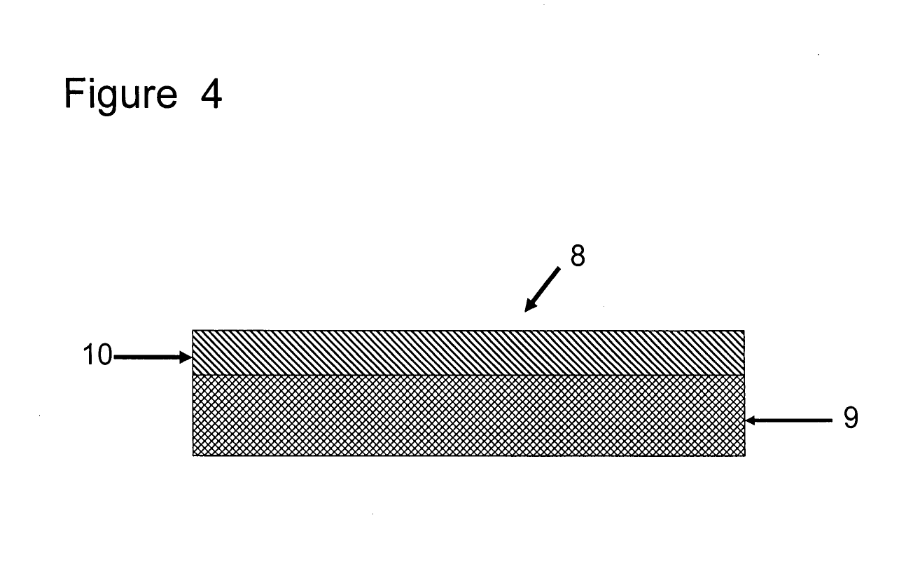

The continuous polymer layer 10 is preferably an elastomer, such as but not limited to, aromatic and aliphatic polyurethanes including copolymers, styrene block copolymers, silicones, thermoplastic silicones, fluoro-silicones, fluoroelastomer, THV, and latex. In one embodiment of the present invention, the continuous polymer layer 10 is coated onto only one side of the porous reinforcing polymer 9, as shown in FIG. 4. As depicted in FIG. 3, the continuous polymer layer 10 can be coated onto both sides of the porous reinforcing polymer 9. In a preferred embodiment as depicted in FIG. 4, the continuous polymer layer 10 can be imbibed into the porous reinforcing polymer 9 and the imbibed polymer 11 fills some or all of the pores of the porous reinforcing polymer 9. The continuous polymer layer 10 of the present invention is coated onto at least one side of the porous reinforcing polymer 9 as depicted in FIGS. 3 and 4. The continuous polymer layer can be applied to the porous reinforcing polymer through any number of conventional methods including but not limited to, lamination, transfer roll coating, wire-wound bar coating, reverse roll coating, and solution coating or solution imbibing. In a preferred embodiment, the continuous polymer layer is solution imbibed into at least one side of the porous reinforcing polymer as depicted in FIG. 5, and forms a continuous polymer layer 10 on both sides of the porous reinforcing polymer 9. In this embodiment, the continuous polymer layer polymer is dissolved in a suitable solvent and coated onto and throughout the porous reinforcing polymer using a wire-wound rod process. The coated porous reinforcing polymer is then passed through a solvent oven and the solvent is removed leaving a continuous polymer layer coated onto and throughout the porous reinforcing polymer. In some cases, such as when silicone is used as the continuous polymer layer, the coated porous reinforcing polymer may not require the removal of solvent. In another embodiment, the continuous polymer layer is coated onto at least one side of the porous reinforcing polymer and maintained in an uncured state where it can be subsequently cured. For example an ultraviolet light (UV) curable urethane may be used as the continuous polymer layer and coated onto the porous reinforcing polymer. The composite film comprising the porous reinforcing polymer and the UV curable urethane continuous polymer layer can then be wrapped around the tube, formed into a shape and then exposed to UV light and cured.

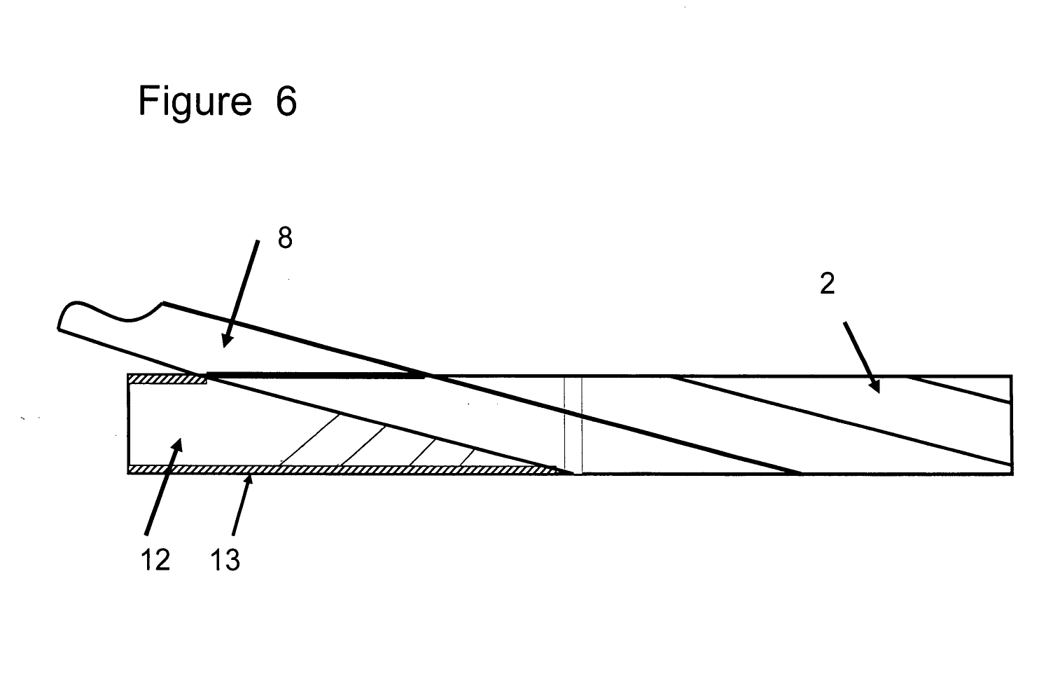

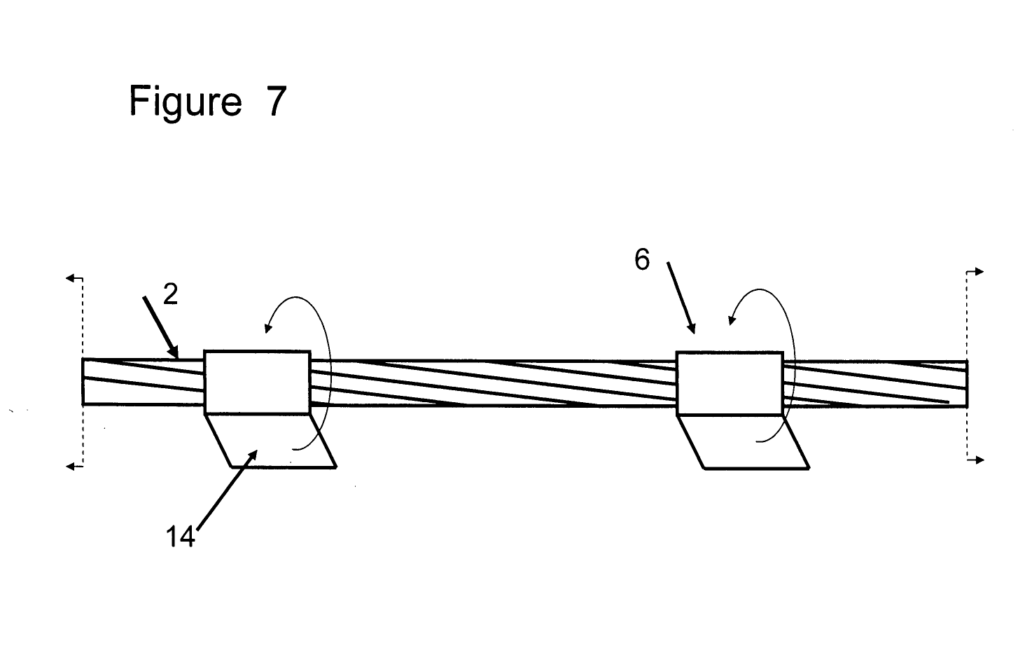

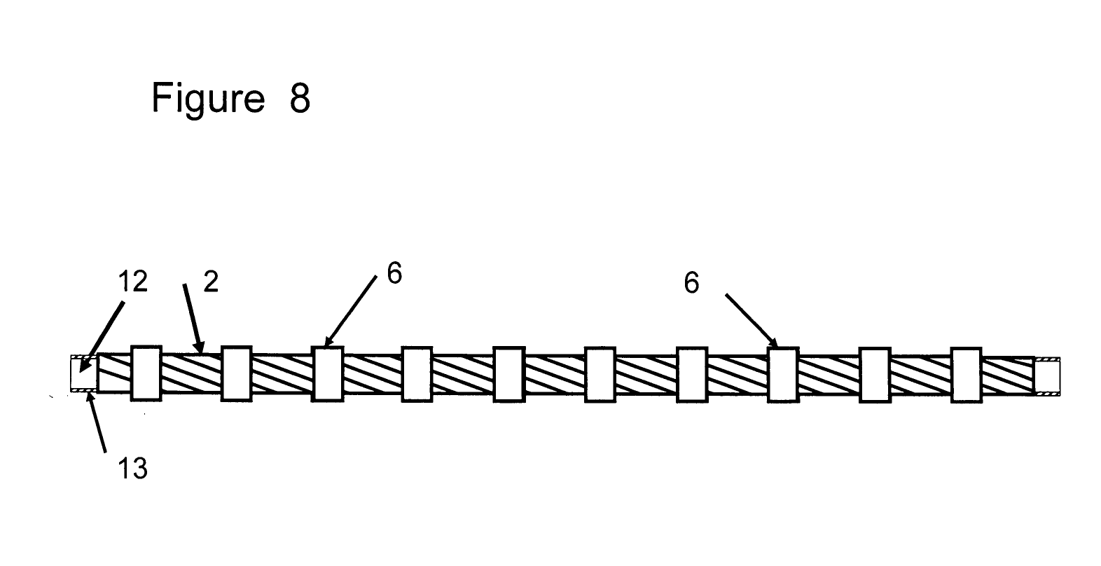

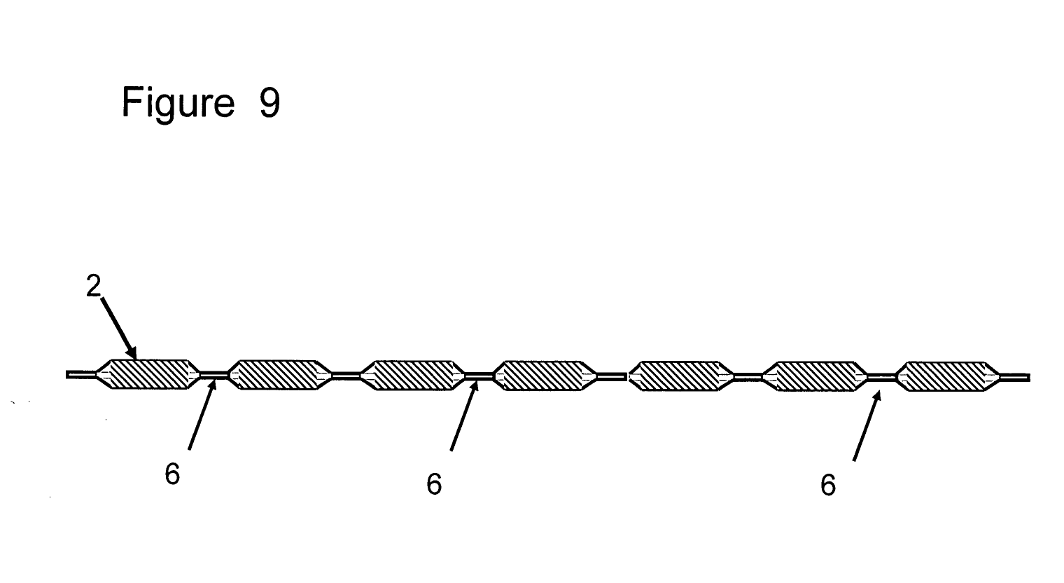

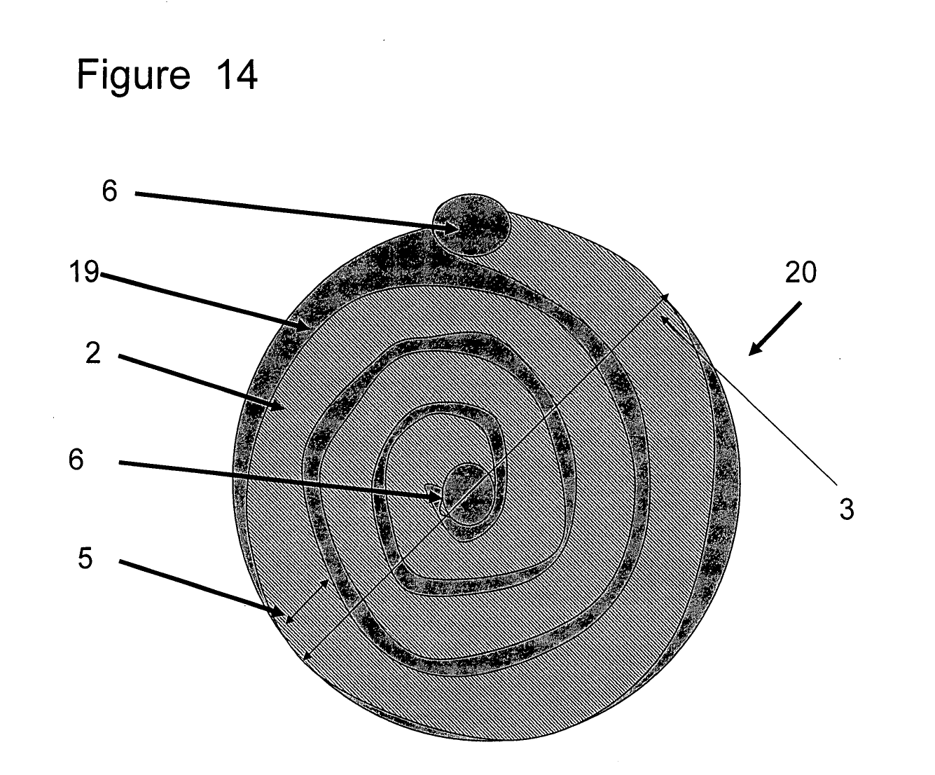

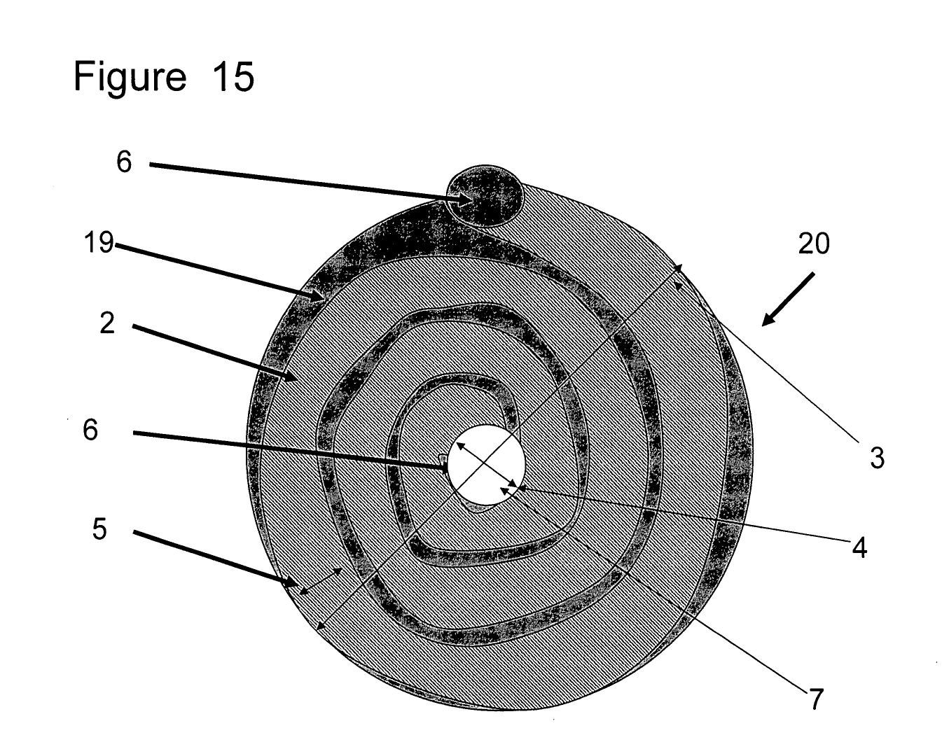

In this preferred embodiment, the ePTFE membrane is anisotropic such that it is highly oriented in the one direction. An ePTFE membrane with a matrix tensile value in one direction of greater than 690 megapascals is preferred, and greater than 960 megapascals is even more preferred, and greater than 1,200 megapascals is most preferred. The exceptionally high matrix tensile value of ePTFE membrane allows the composite material to withstand very high hoop stress in the inflated balloon configuration. In addition, the high matrix tensile value of the ePTFE membrane makes it possible for very thin layers to be used which aid in a reduced balloon profile. As depicted in FIG. 6, at least two passes of the composite film 8 are wrapped around a core wire 12 in opposing orientations to the longitudinal axis to comprise the first balloon material. The core wire may be coated or treated with a release layer 13, such as fluorinated ethylene propylene (FEP) on the outside surface of the core wire 12. The composite film 8 is preferably helically wrapped around the core wire 12 at an angle of less than 55 degrees from the longitudinal axis of the core wire as measured prior to inflation. After the wire has been wrapped with the composite film 8, the wrapped passes 2 are heated to bond the helically wrapped layers together. Any suitable means can be used to bond the helically wrapped layers together, such as heat, ultrasonic welding, or adhesives. After the helically wrapped layers are bonded, second balloon material layer 14 as shown in FIG. 7 is wrapped over the first balloon material at approximately 90 degrees from the longitudinal axis of the core wire 12. A composite film comprising the same or different types of materials can be used for the second balloon material 14. The second balloon material forms a non-distensible region 6 which remains relatively stationary during inflation as is also depicted in FIGS. 8 and 9. The high angle of the wrapped balloon passes and the high strength of the second balloon material limit distension in these over-wrapped regions forming non-distensible regions 6. A release layer 13 is shown covering the core wire 12 for easy release of the balloons. Non-distensible regions 6 aid in forming the inflatable shaped balloon 1. In a folded shaped balloon configuration as depicted in FIGS. 1, 2, 10, 11, 12, and 13, a number of non-distensible regions 6 are formed and connect the inflated portions of the folded multi-lobed shaped balloon. In the spiral shaped balloon 20 configuration as depicted in FIGS. 14 and 15, the balloons are comprised of wrapped balloon passes 2 with non-distensible regions 6 formed at the ends of the balloon. After the second balloon material is over-wrapped, it is then bonded to the other wrapped balloon layers using any number of suitable means such as heat, ultrasonic welding, or adhesives. With the non-distensible regions formed, the core wire and the slip layer are removed by cutting away a section of the helically wrapped composite film from the ends of the wire and stretching the wire and slip layer to remove them. A segmented, hollow balloon tube is produced.

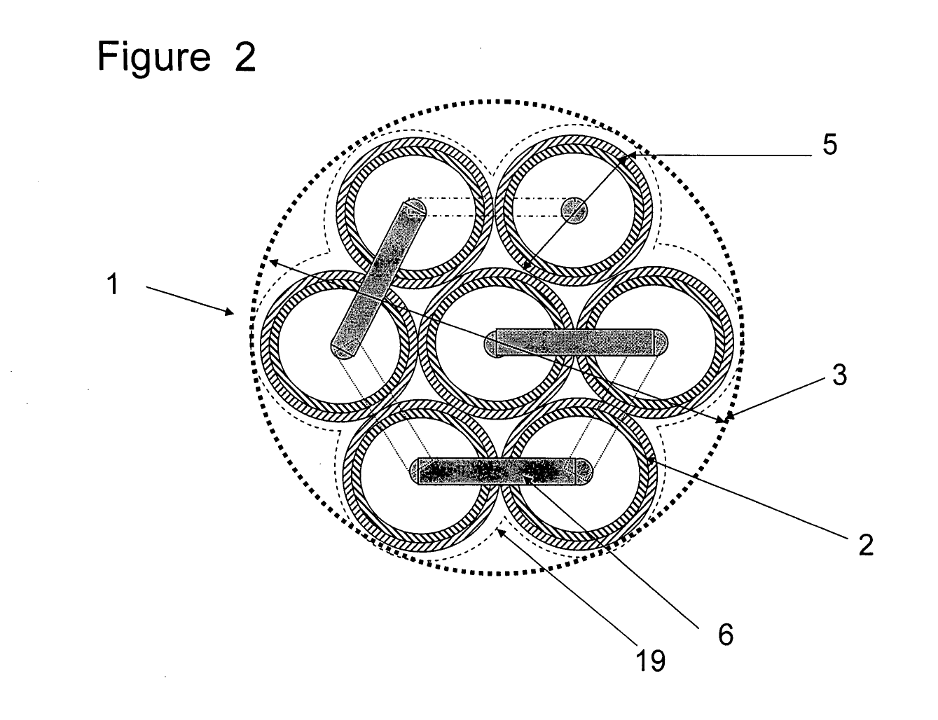

The segmented, hollow balloon tube is then formed into a shape and fixed into that shape through heating or over-wrapping with an outer configuration layer(s) 19 as depicted in FIGS. 1, 2, 10, 11 and 14-18. In one embodiment, one end of the hollow balloon tube is attached to a catheter and the other is sealed closed. The hollow balloon tube is then inflated and subsequently folded into a shaped balloon as depicted in FIG. 2. One or more outer configuration layers 19 may be wrapped at a similar angle to the longitudinal axis of the shaped balloon. In this embodiment, an outer configuration layer 19 is wrapped around the folded shaped balloon and bonded. The configuration layer bonds the helically wrapped tube together in a desired form. The outer configuration layer 19 is preferably a composite film 8 as depicted in FIGS. 3 to 5 and has enough mechanical strength to secure the inflated shaped balloon in the predetermined shape without appreciable distention or expansion. In addition, it is preferable that a very thin and strong composite film be used as the outer configuration layer to reduce the number of over-wrap layers needed for securing the shape and to minimize the diameter of the shaped balloon. It is preferred to use a material for the outer configuration layer, which prevents shortening of the balloon upon inflation.

In another embodiment, the hollow balloon tube is wound into a spiral wound shaped balloon 20 and fixed into that shape through heating or over-wrapping with an outer configuration layer 19 as depicted in FIG. 14. In this embodiment, one end of the hollow balloon tube is attached to a catheter and the other is sealed closed, and the hollow balloon tube is then inflated and subsequently spiral wound and over-wrapped with an outer configuration layer. FIG. 14 depicts the shaped balloon presenting a larger outer diameter 3 achieved by this balloon as compared to the inflated balloon diameter 5. As depicted in FIG. 15, an open region 7 may be formed in at the center of the spiral wound shaped balloon 20. FIG. 15 depicts the wrapped balloon passes 2 formed into a tube shaped balloon with the inflated balloon diameter 5 being much less than the outer diameter 3 of the spiral shaped balloon.

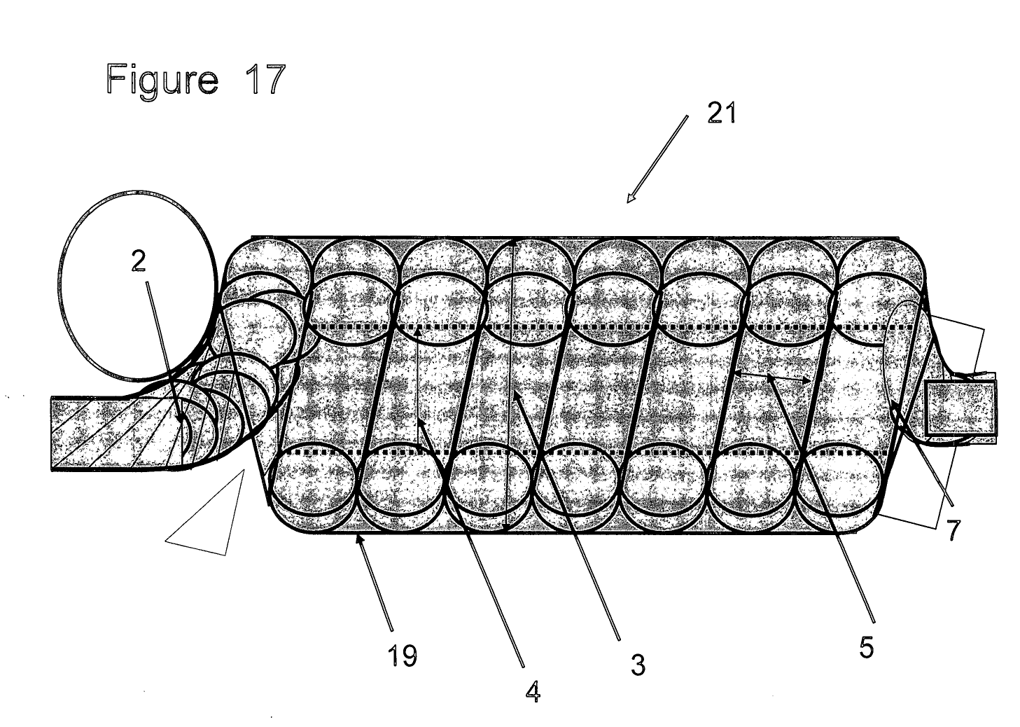

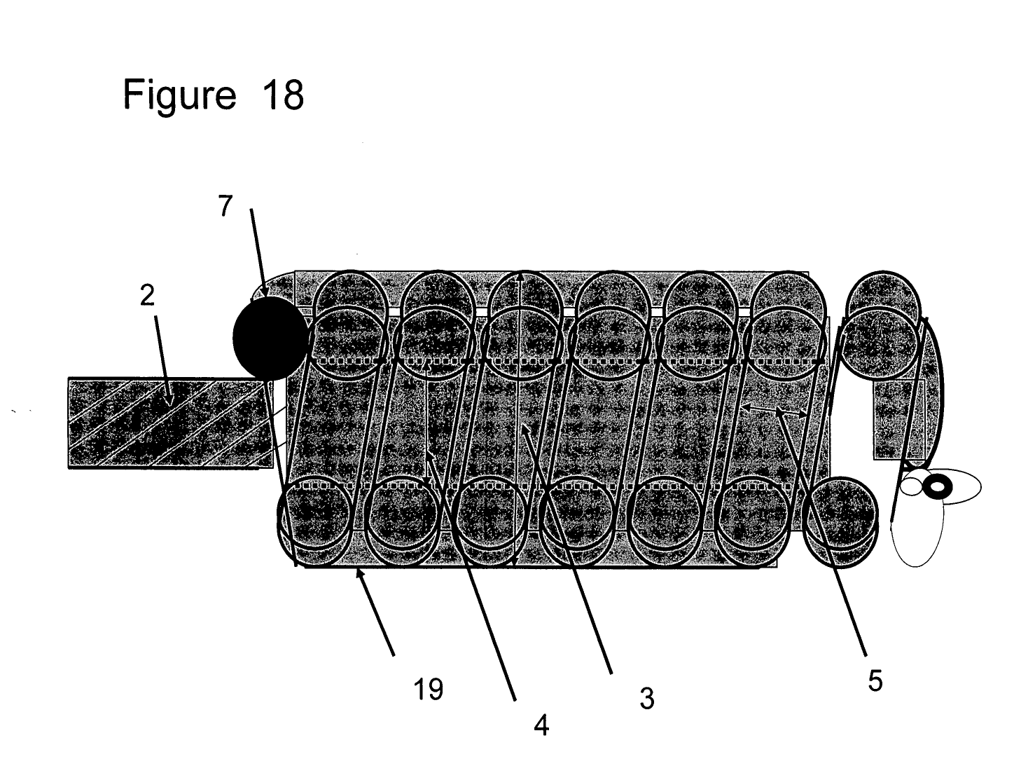

In another embodiment, the hollow balloon tube is formed into a coil shaped balloon 21 around a center axis and fixed into that shape through heating or over-wrapping with an outer configuration layer 19 as depicted in FIG. 17. In this configuration, the hollow balloon tube can be coiled around a mandrel and fixed into that shape and the mandrel can subsequently be removed, leaving an open region 7 as depicted in FIG. 17. In another embodiment, the hollow balloon tube can be coiled around itself, with a portion of the hollow balloon tube defining the central axis as depicted in FIG. 18.

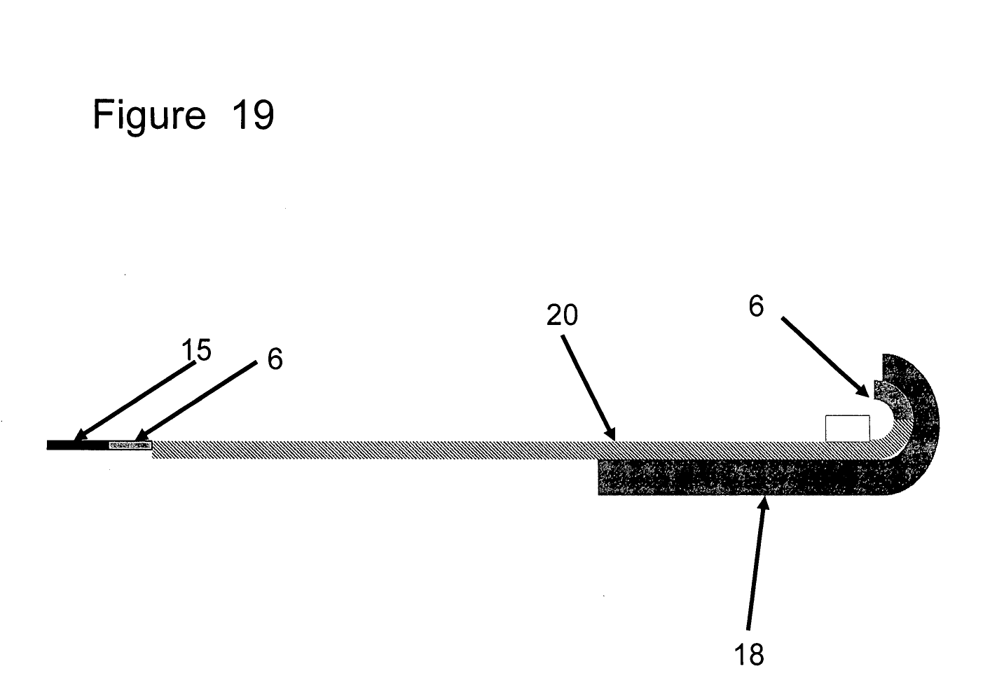

In yet another embodiment, a portion of the length of the hollow balloon tube is attached to a treatment device 18 and subsequently spiral wound as depicted in FIG. 19. In this embodiment, the spiral shaped balloon 20, with a non-distensible region 6 attached to a catheter 15, can be used to deploy or place the treatment device 18 in a desired location through inflation and unfurling of the balloon. Means may be employed to detach the treatment device from the shaped balloon.

A small profile is necessary for the balloon to be able to be positioned in small arteries or veins or orifices. In order for balloons to be positioned in some areas of the body, the balloon catheter must be able to move through a small bend radius, and a thinner walled tube is typically much more supple and capable of bending in this manner without creasing or causing damage to the wall of the vessel.

In another embodiment, the ePTFE membrane used in the configuration layers are relatively mechanically isotropic or homogeneous. The mechanically balanced ePTFE membrane can increase the maximum hoop stress that the composite film made therefrom can withstand, such as discussed in Example 3.

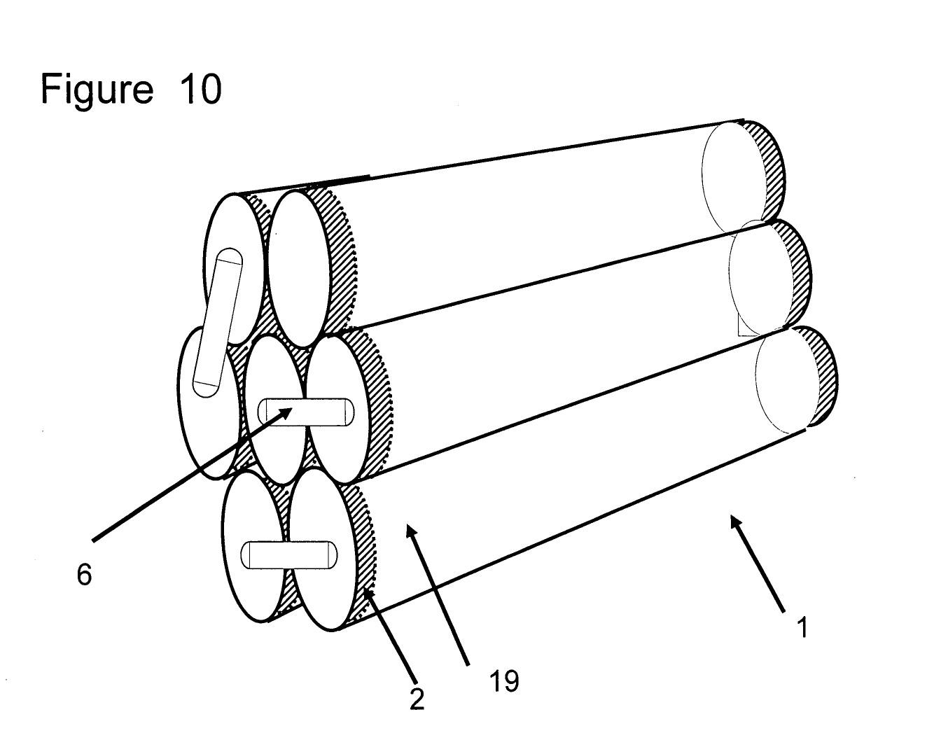

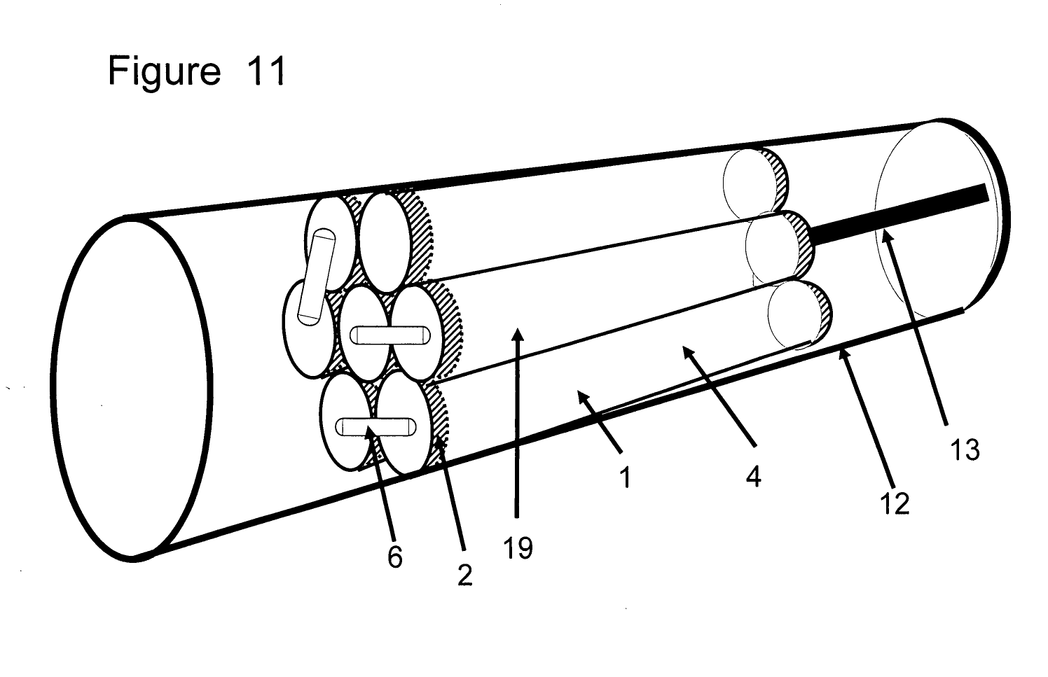

The inflated shaped balloons 1 of the present invention comprise wrapped balloon layers 2 and non-distensible regions 6 and can be further reinforced with the addition of an outer configuration layer 19 as depicted in FIGS. 10 to 14. The outer configuration layer 19 can be a composite film, or a porous reinforcing polymer as described in the present invention, and can be attached to the balloon through any number of conventional methods including but not limited to, adhesion, heat sealing, UV curing, and ultrasonic welding. In a preferred embodiment, the outer configuration layer is made with ePTFE membrane. A preferred coating is urethane. The balloons may comprise non-distensible regions 6. FIGS. 11, 12, and 13 show the inflated shaped balloon as a component of a catheter 15 inside of a vessel 16. FIG. 12 further shows a perfusive flow region 17 through the center of the inflated shaped balloon 1 to alleviate pressure on the vessel 16.

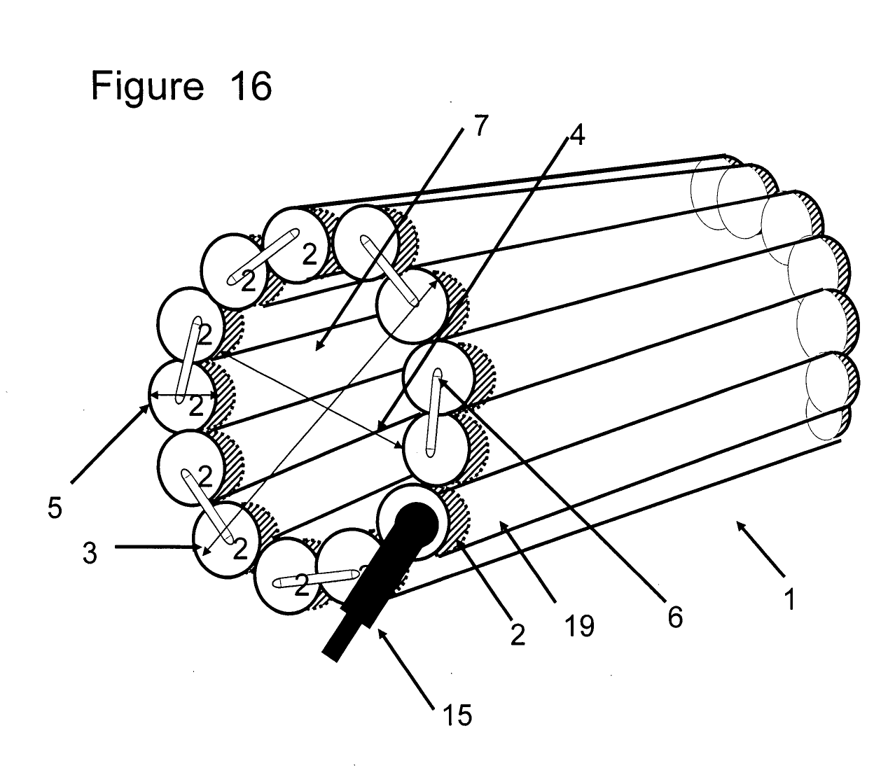

The shaped balloon of the present invention can withstand high inflation pressures relative to the outer diameter achieved due to the outer diameter 3 being larger than the inflated balloon diameter 5, as shown in FIG. 1. Thus, the maximum hoop stress of the inflated balloons of the present invention are much lower than those of conventional cylindrical angioplasty balloons at similar outer diameters. The porous reinforcing polymer greatly increases the maximum hoop stress and allows the balloon to maintain a shape in an inflated state under high inflation pressure. The hoop stress is proportional to the product of the pressure of inflation and the balloon diameter 3, divided by the wall thickness of the inflated balloon. For a given inflation pressure, the shaped balloons of the present invention can be constructed to have much larger outer diameters 3 than a conventional tubular balloon made of the same material. In a preferred embodiment, the outer diameter 3 of the balloon is greater than 1.5 times the inflated balloon diameter 5 as depicted in FIG. 1. In a more preferred embodiment, the outer diameter 3 is greater than 3.0 times the inflated balloon diameter, and in the most preferred embodiment, the outer diameter 3 is greater than 5.0 times the inflated balloon diameter as depicted in FIGS. 16 and 17. In a preferred embodiment, a high matrix tensile strength ePTFE membrane is used as the porous reinforcing polymer and an inflated balloon with an outer diameter 3 of greater than 6 mm is made to withstand a hoop stress of greater than 400 megapascals, and more preferred is made to withstand a hoop stress of greater than 600 megapascals.

The balloons of the present invention can be attached to a catheter through any number of conventional means. In a preferred embodiment as depicted in FIGS. 11 to 13, and 15, excess length of the wrapped balloon layers are used to seal the balloon to the catheter. Additional wraps of composite film or porous reinforcing polymer can be used to further increase the bond to the catheter.

The balloons of the present invention, when attached to a catheter, are capable for use in various surgical procedures including but not limited to angioplasty, stent or graft delivery and distention, valvuloplasty, aortic stent graft deployment, pediatric coarctation, sizing balloon for ASD/PFO, endocardial procedures, stent deployment, temporary brachytherapy, vertebrae compression and compaction, as well as intestinal procedures. The shaped balloons of the present invention are particularly useful in procedures requiring a large diameter elastomeric balloon catheter. In a preferred embodiment, the balloon is made with an open region 7 that allows for flow 17 through the inflated shaped balloon 1, deployed in a vessel 16 as depicted in FIG. 12. In yet another embodiment as depicted in FIG. 11, the open region is eliminated and the region within the inner diameter 4 of the inflated shaped balloon 1 is completely sealed to prevent any flow through the balloon. The open region can be sealed by the folding configuration or an outer configuration layer of material may be used to encapsulate the entire inflated balloon.

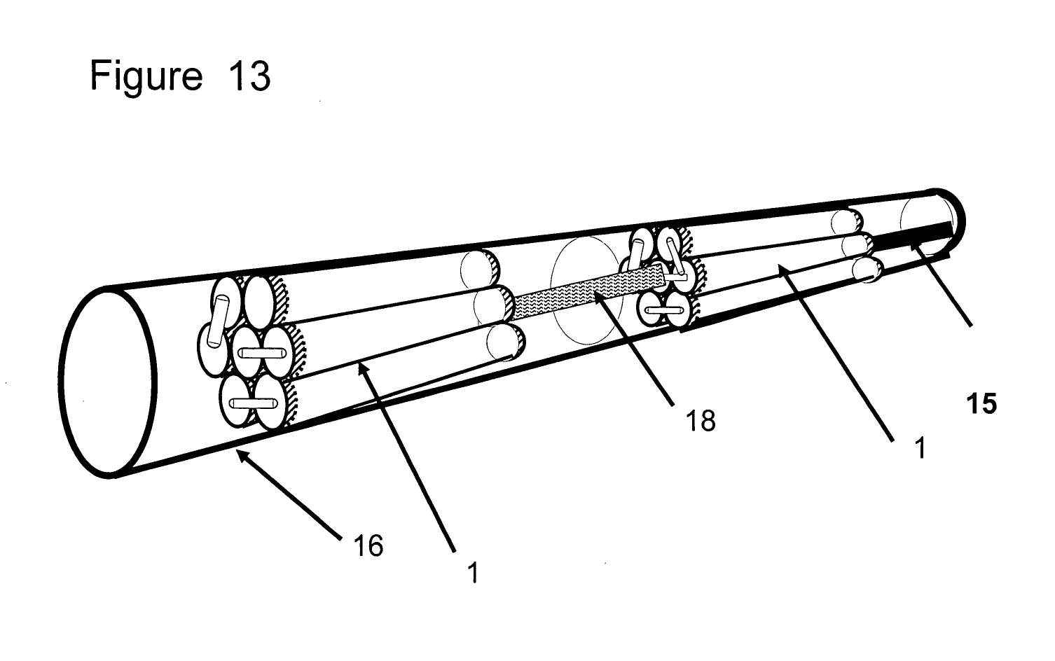

In yet another embodiment of the present invention as depicted in FIG. 13, a treatment element 18 is secured between two inflated folded shaped balloons 1, wherein the treatment element is located approximately in the center of the vessel 16. In this embodiment the treatment element may be positioned between the two inflated balloons through the catheter 15. The ability to secure a treatment element in the center of a vessel or orifice is especially valuable when radioactive materials are used as the treatment element such as temporary brachytherapy procedures. The inflated shaped balloon can be used to a planar item in a vessel or body. When attached to a planar item in a manner such that the planar item may be folded and compressed prior to inflation, the shaped balloon inflates and restores the sheet-like item to its original planar form.

In another embodiment of the present invention, the open region 7 can be made to close upon inflation of the balloon. This would allow the balloon to be positioned in a vessel and then decrease flow upon inflation. In a preferred embodiment, the balloon is used to control the flow rate through a vessel through the use of inflation pressure. In yet another embodiment, the shaped balloon of the present invention is used to increase the flow through a vessel. When increased flow in a vessel is desired, the shaped balloon of the present invention is configured with an open region and is positioned in the vessel and inflated. The shaped balloon expands and increases the diameter of the vessel allowing an increased flow through the open region 7. In another embodiment, the balloon with a closing open region 7 during inflation is used to secure devices or tissue for placement, delivery into or removal from the body.

In another embodiment of the present invention, bioresorbable polymer is used as the porous reinforcing polymer in the construction of the balloon. Bioresorbable polymer can also be used as the continuous polymer layer and would leave behind the porous reinforcing polymer after being absorbed by the body. This use of the present invention may be of particular value in intestinal or abdominal hernia applications, or aneurysm applications. In yet another embodiment of the present invention, a bioresorbable polymer is used in the construction of the composite film and is used as an inflation fluid to deploy the balloon.

In another embodiment, the balloon of the present invention can be made to detach from the catheter after location in the body, and subsequent inflation. In this embodiment it is preferred that the composite film be made to be self sealing such that the balloon stays inflated after removal of the inflation tube. In another embodiment, the inflation tube can be sealed and the catheter can be made to detach from the inflation tube after locating and inflating the balloon.

In yet another preferred embodiment, the shaped balloon of the present invention is able to realize an inflated outer diameter of at least 10 mm at an inflation pressure of at least 10 atmospheres and maintain the shape profile. In another preferred embodiment, the shaped balloon of the present invention is able to realize an inflated diameter of at least 20 mm, and an axial length of 5 mm at an inflation pressure of at least 10 atmospheres and maintain the shape profile.

While particular embodiments of the present invention have been illustrated and described herein, the present invention should not be limited to such illustrations and descriptions. It should be apparent that changes and modifications may be incorporated and embodied as part of the present invention within the scope of the following claims. The following examples are further offered to illustrate the present invention.

### EXAMPLES

### Example 1

### Test Methods

Tensile break load was measured using an INSTRON 1122 tensile test machine equipped with flat-faced grips and a 0.445 kN load cell. The gauge length was 5.08 cm and the cross head speed was 50.8 cm/min. The sample dimensions were 2.54 cm by 15.24 cm. For longitudinal MTS measurements, the larger dimension of the sample was oriented in the machine, also known as the down web direction. For the transverse MTS measurements, the larger dimension of the sample was oriented perpendicular to the machine direction, also known as the cross web direction. Each sample was weighed using a Mettler Toledo Scale Model AG204, then the thickness of the samples was taken using the Kafer FZ1000/30 thickness gauge. The samples were then tested individually on the tensile tester. Three different sections of each sample were measured. The average of the three maximum load (i.e., the peak force) measurements was used. The longitudinal and transverse MTS were calculated using the following equation:

MTS=(maximum load/cross section area)*(bulk density of PTFE)/density of the porous membrane),

wherein the density of PTFE is taken to be 2.2 g/cc.

### Example 2

### Composite Film and Wire Core

The inflatable balloon of the present invention was made by wrapping a composite film of Tecothane TT-1085A polyurethane (Thermedics, Inc, Woburn, Mass.), and ePTFE membrane over a urethane-coated Tefzel core wire (Putnam Plastics LLC, Dayville, Conn.). The wrapped core wire was heat treated and the center wire were subsequently removed to provide a hollow composite balloon tube.

The core wire was a (nominal) 0.74 mm diameter Tefzel 280 wire with a (nominal) 0.05 mm Tecothane TT-1074A coating. The ePTFE membrane used to make the composite film was made in accordance with the teaching in U.S. Pat. No. 5,476,589 to Bacino, incorporated herein by reference. Specifically, the ePTFE membrane was longitudinally expanded to a ratio of 55 to 1 and transversely expanded approximately 2.25 to 1, to produce a thin strong membrane with a mass of approximately 3.5 g/m² and a thickness of approximately 6.5 micrometers.

The composite film was made by using a wire-wound rod coating process whereby a solution of Tecothane TT-1085A polyurethane and tetrahydrofuran (THF) was coated onto an ePTFE membrane. A 3% to 8% by weight solution of Tecothane TT-1085A polyurethane in THF was coated onto the ePTFE membrane to produce a composite film with approximately equal amounts of Tecothane TT-1085A polyurethane on either side and throughout the ePTFE membrane and a total polymer weight application of approximately 40% to 60% of the total final composite film weight.

### Example 3

The composite film described in Example 1 was slit to 12 mm wide and helically wrapped around the core wire described in Example 1 at a 4 to 5 degree angle from the longitudinal axis of the wire. The wrapped core wire was heated for approximately 5 to 30 seconds at 180° C. after wrapping. The core wire was then wrapped with the composite film in the opposite direction at a 4 to 5 degree angle from the longitudinal axis of the wire and subsequently heated for approximately 5 to 30 seconds at 180° C. The process of wrapping the core wire with this first wrap material in opposite directions and heating after each pass was repeated until a total of twelve passes of wrapping was complete. A second wrap material was made from a mechanically balanced composite film using a wire-wound rod coating process whereby a solution of Tecothane TT-1085A polyurethane and tetrahydrofuran (THF) was coated onto an ePTFE membrane. The ePTFE membrane used to make the composite film was made in accordance with the teachings of U.S. patent application Ser. No. 11/334,243 to Bacino et al. incorporated herein by reference. Specifically, the ePTFE membrane was longitudinally expanded to a ratio of 15 to 1 and transversely expanded approximately 28 to 1, to produce a thin strong membrane with a mass of approximately 3.5 g/m² and a thickness of approximately 8 micrometers. A 3% to 8% by weight solution of Tecothane TT-1085A polyurethane in THF was coated onto the ePTFE membrane to produce a composite film with Tecothane TT-1085A polyurethane on one side of the ePTFE membrane and throughout of the ePTFE membrane, and a total polymer weight application of approximately 40% to 60% of the total final composite film weight. This second wrap layer material was slit to approximately 7.6 mm wide.

The wrapped core was mounted onto a wrapper station, comprising chucks for clamping the ends of the wire, and a variable speed rotation control. The 7.6 mm wide second wrap layer material was wrapped at approximately 90 degrees from the longitudinal axis of the wrapped wire, in eight locations 36 mm apart from each other. The second layer wrap material was wrapped approximately ten times revolutions around the circumference of the wrapped wire to produce an approximately 10 mm wide over-wrapped section. The non-distensible regions were then heated using a Weller WSD81 solder gun (Cooper Industries, Inc. Raleigh, N.C.), with a large blunt solder tip heated to a set-point of 315. The wrapped wire was slowly rotated and the blunt solder tip was pressed onto the over-wrapped non-distensible regions.

The core wire was removed from the composite balloon construction. Approximately a 2.54 cm long section of the composite hollow balloon tube was removed from either end of a 30.5 cm long section of the balloon over wire construction. The exposed ends of the wire were clamped with hemostats and pulled by hand until the wire had been stretched approximately 30 cm, at which point it was removed from the center of the tube. A composite hollow balloon tube was produced with a first layer helically wrapped composite film wrapped at a low (4 to 5 degree) angle of wrap and six 10 mm wide over-wrapped sections with 36 mm spaces between them and two non-distensible seals at the end.

One end of the composite hollow balloon tube was tied into a knot and clamped with a hemostat and the opposite end was slipped through a Qosina male touhy borst with spin lock fitting (#80343, Qosina Corporation, Edgewood, N.Y.), and a Monoject blunt needle with Aluminum luer lock hub (model # 8881-202389, Sherwood Medical, St. Louis, Mo.) was inserted approximately 2.0 cm into the balloon. The hemostatic valve was tightened to seal the balloon, and was then attached to an Encore 26 inflation device (Boston Scientific Scimed, Maple Grove, Minn., catalog #15-105) and inflated to approximately 18 atmospheres with saline solution. The inflation created seven balloon sections or segments separated by non-distensible regions.

The inflated balloon sections were then folded or over-layed with the first balloon section in the center and the remaining six around the circumference of the first section as depicted in FIGS. 1, 9, and 10. The inflated folded balloon was then over-wrapped with 2 wraps of a 25 mm wide second wrap layer material at approximately a 90 degree angle from the longitudinal axis of the composite balloon to form the outer configuration layer. The over-wrapped folded balloon was then heated using a Weller WSD81 solder gun (Cooper Industries, Inc. Raleigh, N.C.), with a large blunt solder tip heated to a set-point of 315. The blunt solder tip was pressed gently against the wrapped outer configuration layer of the folded composite balloon and run along the length of the balloon. This process produced an approximately 25 mm long by approximately 12 to 15 mm diameter balloon.

### Example 4

The composite film described in Example 1 was slit to nominally 23 mm wide and helically wrapped around the core wire described in Example 1 at a 10 to 12 degree angle from the longitudinal axis of the wire. The wrapped core wire was heated for approximately 5 to 30 seconds at 180° C. after wrapping. The core wire was then wrapped with the composite film in the opposite direction at a 10 to 12 degree angle from the longitudinal axis of the wire and subsequently heated for approximately 5 to 30 seconds at 180° C. The process of wrapping the core wire in opposite directions and heating after each pass was repeated until a total of four passes of wrapping was complete. The wrapped core wire was then wrapped around a pin frame with approximately 30 cm spaces between pins and approximately 180 degrees of wrap around each pin and tied at the ends before being placed into an oven and heated for approximately 30 minutes at 150° C.

The core wire was removed from the composite balloon construction. Approximately a 2.54 cm long section of the composite hollow balloon tube was removed from either end of a 30.5 cm long section of the balloon over wire construction. The exposed ends of the wire were clamped with hemostats and pulled by hand until the wire had been stretched approximately 30 cm, at which point it was removed from the center of the tube. A composite hollow inflatable balloon resulted. One end of the composite hollow balloon tube was closed with a knot, and the other end was inserted with a Monoject blunt needle and secured to a Encore 26 inflation device (Boston Scientific Scimed, Maple Grove, Minn., catalog #15-105) via Qosina male touhy borst.

The balloon was inflated and, starting from the center-point, manually coiled into a planar disc-shape having an inflated outer diameter of 1.5 inches and height of 3.5 mm. The coiled, inflated balloon was then over-wrapped with a second wrap layer to form the outer configuration layer. This second wrap material was made from a mechanically balanced composite film using a wire-wound rod coating process whereby a solution of Tecothane TT-1085A polyurethane and tetrahydrofuran (THF) was coated onto an ePTFE membrane. The ePTFE membrane used to make the composite film was made in accordance with the teachings of U.S. patent application Ser. No. 11/334,243 to Bacino et al. incorporated herein by reference. Specifically, the ePTFE membrane was longitudinally expanded to a ratio of 15 to 1 and transversely expanded approximately 28 to 1, to produce a thin strong membrane with a mass of approximately 3.5 g/m² and a thickness of approximately 8 micrometers. A 3% to 8% by weight solution of Tecothane TT-1085A polyurethane in THF was coated onto the ePTFE membrane to produce a composite film with Tecothane TT-1085A polyurethane on one side of the ePTFE membrane and throughout of the ePTFE membrane, and a total polymer weight application of approximately 40% to 60% of the total final composite film weight.

The coiled, inflated, over-wrapped balloon was then heated using a Weller WSD81 solder gun (Cooper Industries, Inc. Raleigh, N.C.), with a large blunt solder tip heated to a set-point of 315. The blunt solder tip was pressed gently against the wrapped outer configuration layer of the balloon and traversed along the balloon surface. This process produced a disc shaped balloon with approximately a 38 mm outer diameter and approximately 3.5 mm in height.

What is claimed is:

1. An inflatable device comprising at least two helically wrapped passes forming a non-configured tube which is configured and joined into an inflatable form, wherein one of the at least two helically wrapped passes is oriented at an angle of less than or equal to about 55 degrees, and one of the at least two helically wrapped passes is oriented at an angle of greater than or equal to about 55 degrees.

2. The inflatable device of claim 1 wherein the helically wrapped passes comprise a porous reinforcing layer and a continuous polymer layer.

3. The inflatable device of claim 1 further comprising an outer configuration layer which fixes the non-configured tube into an inflatable form.

4. The inflatable device of claim 1 wherein the at least two helically wrapped passes are anisotropic and the outer configuration layer is anisotropic.

5. The inflatable device of claim 1 wherein the helical wrap layers are anisotropic and the outer configuration layer is isotropic.

6. An inflatable device comprising at least two helically wrapped passes and at least one configuration layer forming a tube and an inflation means connected to said tube, wherein the tube is configured and joined in at least one area forming an inflatable device with multiple cross-sectional segmented areas.

7. The inflatable device of claim 6 wherein one of the one of the two helically wrapped passes is oriented at an angle of less than or equal to about 55 degrees, and one of the at least two helically wrapped passes is oriented at an angle of greater than or equal to about 55 degrees.

8. The inflatable device of claim 6 further comprising a non-distensible region.

9. The inflatable device of claim 8 wherein the two passes and the non-distensible region are continuously wrapped.

10. The inflatable device of claim 6 wherein the tube is configured into a folded multi-lobed shape around a center axis.

11. The inflatable device of claim 9 wherein the non-distensible regions are located at the foldover points of the tube.

12. The inflatable device of claim 6 wherein the tube is configured into a spiral wound flat disc shape.

13. The inflatable device of claim 6 wherein the tube is configured into a coiled shape around a center axis.

14. The inflatable device of claim 6 further comprising an open region.

15. The inflatable device of claim 6 further comprising an outer configuration layer.

16. The inflatable device of claim 6 configured in the form of a multi-lobed balloon suitable for bifurcation.

17. The inflatable device of claim 6 configured in the form of a multi-lobed balloon suitable for trifurcation.

18. The inflatable device of claim 6 configured in the form of a sequential multi-lobed balloon wherein the lobes are interconnected via a single, common inflation lumen.

19. The inflatable device of claim 6 wherein the inflatable device has an un-inflated length which remains relatively unchanged upon inflation.

20. The inflatable device of claim 6 wherein the at the least two helically wrapped inner layers comprise a fibrous reinforcement.

21. The inflatable device of claim 6 wherein the at least two helically wrapped inner layers are comprised of a porous reinforcing polymer.

22. The inflatable device of claim 6 wherein the at least two helically wrapped layers are comprised of a porous reinforcing polymer and a continuous phase of polymer.

23. The inflatable device of claim 21 wherein the porous reinforcing polymer is an olefin.

24. The inflatable device of claim 21 wherein the porous reinforcing polymer comprises an anisotropic polymer.

25. The inflatable device of claim 21 wherein the porous reinforcing polymer comprises PEEK.

26. The inflatable device of claim 21 wherein the porous reinforcing polymer comprises a polyamide.

27. The inflatable device of claim 21 wherein the porous reinforcing polymer is a polyurethane.

28. The inflatable device of claim 21 wherein the porous reinforcing polymer is a polyester.

29. The inflatable device of claim 21 wherein the porous reinforcing polymer comprises a fluoropolymer.

30. The inflatable device of claim 29 wherein the fluoropolymer is expanded PTFE.

31. The balloon of claim 30 wherein the expanded PTFE has a matrix tensile value in one direction of greater than 690 megapascals.

32. The balloon of claim 30 wherein the expanded PTFE has a matrix tensile value in one direction of greater than 960 megapascals.

33. The balloon of claim 30 wherein the expanded PTFE has a matrix tensile value in one direction of greater than 1,200 megapascals.

34. The balloon of claim 30 wherein the maximum hoop stress of the helically wrapped layers is greater than 400 megapascals.

35. The balloon of claim 30 wherein the maximum hoop stress of the helically wrapped layers is greater than 600 megapascals.

36. The inflatable device of claim 22 wherein the continuous phase of a polymer is a fluoropolymer.

37. The inflatable device of claim 22 wherein the continuous phase of a polymer is an elastomer.

38. The inflatable device of claim 22 wherein the continuous phase of a polymer is a urethane.

39. The inflatable device of claim 22 wherein the continuous phase of a polymer is a silicone.

40. The inflatable device of claim 22 wherein the continuous phase of a polymer is a fluoro-elastomer.

41. The inflatable device of claim 19 wherein the continuous phase of a polymer is bioresorbable.

42. A deployment mechanism for a planar item comprising the inflatable device of claim 8 attached to a planar item in a manner such that the planar item may be folded and compressed prior to inflation so that upon inflation, the balloon inflates and restores the sheet-like item to its original planar form.

43. The balloon of claim 6 further comprising a treatment element.

44. The balloon of claim 43 wherein the treatment element is radioactive.

45. A method of making an inflatable shaped balloon comprising the steps of:
helically wrapping at least two layers of composite film around a tube;
applying heat to the helically wrapped layers, bonding the helically wrapped layers together;
attaching the bonded helically wrapped layer to an inflation means;
inflating the helically wrapped layers and configuring the balloon into a shape; and
fixing the configured balloon in said shape.

46. The method of claim 45 wherein said shape is a folded multi-lobed shape around a center axis.

47. The method of claim 45 wherein the said shape is a spiral wound shape.

48. The method of 45 wherein the said shape is a coiled shape around a center axis.

49. The method of claim 45 wherein the composite film comprises a porous reinforcing polymer and a continuous polymer layer.

50. The method of claim 49 wherein the porous reinforcing polymer is ePTFE.

51. A method of controlling the flow through a vessel comprising the steps of
positioning the shaped balloon in a desired location in a vessel; and
inflating the balloon to a desired pressure thereby reducing the open area and reducing the flow through the vessel.

52. The method of claim 51 wherein the vessel is a blood vessel.

53. The method of claim 51 wherein the flow is blood flow.

54. A method of controlling the flow through a vessel comprising the steps of
positioning the hollow-centered balloon in a desired location in a vessel; and
inflating the balloon to a desired pressure thereby increasing the diameter of the vessel and increasing the open area and increasing flow through the vessel.

55. The method of claim 54 wherein the vessel is a blood vessel.

56. The method of claim 54 wherein the flow is blood flow.
